(19) United States
(12) Patent Application Publication
(10) Pub. No.: US 2024/0176023 A1
(43) Pub. Date: May 30, 2024

(54) ADJUSTMENT OF FREQUENCY OF A MOTION MEASURING SYSTEM

(71) Applicant: Sword Health, S.A., Porto (PT)

(72) Inventors: Pedro Miguel Simoes Bastos Martins, Porto (PT); Pedro Miguel Moreira de Sousa, Porto (PT); José Carlos Coelho Alves, Porto (PT); João Paulo Dias Andrade, Porto (PT); Márcio Filipe Moutinho Colunas, Porto (PT); Luís António Correia de Oliveira, Porto (PT); Virgílio António Ferro Bento, Porto (PT)

(21) Appl. No.: 18/516,784

(22) Filed: Nov. 21, 2023

(30) Foreign Application Priority Data
Nov. 25, 2022 (EP) 22398025.1

(51) Int. Cl. G01S 17/66; A61B 5/0205; A61B 5/08; G01S 7/497

(52) U.S. Cl. CPC G01S 17/66; A61B 5/02055; A61B 5/0816; G01S 7/497

(57) ABSTRACT

A motion measuring system comprises a plurality of sensors and at least one computing device in communication with the plurality of sensors. The plurality of sensors comprise one or more optical sensors and one or more body member sensors. A user interface provided by the at least one computing device is used to instruct a subject to perform a movement of one or more body members. An adjustment of one or more frequencies of one or more of the plurality of sensors is performed to reduce a power consumption of at least one of the plurality of sensors. The adjustment is performed based at least in part on the movement which the subject is instructed to perform. A motion of the subject is measured using the one or more optical sensors of the motion measuring system during or subsequent to performing the adjustment of the one or more frequencies.

### CLAIM OF PRIORITY

This application claims the benefit of priority to European Patent Application No. 22398025.1, filed on Nov. 25, 2022, which is incorporated herein by reference in its entirety.

### TECHNICAL FIELD

The present disclosure relates to the technical field of motion measuring systems.

### BACKGROUND

Many motion measuring systems comprise one or more optical sensors that are arranged at a distance from a target user so that the one or more optical sensors receive light from one or more body members of the user. The one or more optical sensors may provide measurements of light received from the one or more body members. These measurements of light may then be processed by a computing device to derive measurements of motion of the one or more body members.

### BRIEF DESCRIPTION OF THE DRAWINGS

To complete the description and in order to provide for a better understanding of the disclosure, a set of drawings is provided. Said drawings form an integral part of the description and illustrate examples of the disclosure, which should not be interpreted as restricting the scope of the disclosure, but just as examples of how the disclosure can be carried out. The drawings comprise the following figures:

FIG. 1 diagrammatically shows a motion measuring system in accordance with some examples.

FIG. 2 shows a person performing predetermined movements while wearing additional sensors, according to some examples.

FIG. 3 shows a person performing predetermined movements while wearing additional sensors, according to some examples.

### DETAILED DESCRIPTION

In order to enhance an accuracy with which a motion measuring system measures the motion of the one or more body members, it may be advantageous to configure the motion measuring system so that measurements of light are taken, processed, and/or transmitted at a relatively high frequency. However, using a relatively high frequency may cause drawbacks, such as a relatively high power consumption and increased data traffic.

Sensors (e.g., optical sensors) of the motion measuring system may obtain power from, for example, a battery. Therefore, the relatively high consumption of power by the sensors causes an increase in the power obtained from the battery and an increase in the number of charge/discharge cycles of the battery, thereby potentially worsening, among others, durability and performance of the battery.

A motion measuring system configured for sampling, via the sensors of the motion measuring system, at a relatively low frequency could decrease the accuracy of the motion measuring and of estimations based on the measurements resulting from the sampling.

Examples described herein enhance or maintain accuracy of estimations or measurements provided by the motion measuring system, such as accuracy of motion measuring, while minimizing a negative impact on communications and power consumption of the motion measuring system. Examples described herein may reduce a total power consumption of the motion measuring system.

In a first aspect of the disclosure, a method for adjusting one or more frequencies associated with at least one sensor of a motion measuring system is provided. The at least one sensor comprises one or more optical sensors. The one or more optical sensors may be configured to be arranged at a distance from a user so that the one or more optical sensors receive light from one or more body members of the user. The one or more optical sensors may be configured to provide measurements of light received from the one or more body members to measure motion of the one or more body members. The at least one sensor may comprise one or more additional sensors arrangeable on the user.

The method may include configuring at least one frequency associated with the respective sensor of the at least one sensor at least based on a predetermined movement to be performed by the user, thereby modifying a value of at least one predetermined frequency associated with the respective sensor. The at least one predetermined frequency associated with the respective sensor may comprise at least one of: a predetermined measurement frequency with which the respective sensor measures a respective physical magnitude, a predetermined digitizing frequency with which measurements taken by the respective sensor are digitized, a predetermined processing frequency with which the digitized measurements are processed, and a predetermined transmission frequency with which measurements taken by the respective sensor are transmitted to a computing device. The computing device may be configured to receive measurements from the respective sensor.

The at least one frequency to be configured may comprise at least one of: a measurement frequency, a digitizing frequency, a processing frequency, and a transmission frequency.

The motion measuring system may comprise data indicative of a first plurality of adjustment values for configuring the respective at least one frequency based on a predetermined movement to be performed by the user. Each adjustment value of the first plurality of adjustment values may be associated with one predetermined movement to be performed by the user, and each adjustment value of the first plurality of adjustment values may be configured to be applied to the respective predetermined frequency associated with the sensor.

Accordingly, at least one of the measurement frequency, the digitizing frequency, the processing frequency, and the transmission frequency associated with the respective sensor may be configured based on adjustment values of the first plurality of adjustment values. Each adjustment value of the first plurality of adjustment values may be associated with one predetermined movement to be performed by the user.

In some examples, each of the at least one frequency has a corresponding predetermined frequency. The method may configure the at least one frequency associated with the respective sensor based on the predetermined movement to be performed by the user. As a result, the method may allow for increasing and decreasing the at least one frequency for enhancing a tradeoff among high accuracy of the motion measuring and/or of estimations based on measurements of the respective sensor (e.g. an estimation of at least one of: temperature, respiration rate, and pulse rate), low power consumption by the motion measuring system and, if the transmission frequency is adjusted, low congestion of a channel through which the measurements taken by the respective sensor are transmitted to the computing device. In addition, the first aspect of the disclosure may potentially allow for detecting dynamic movements, such as running and jumping.

In some examples, the measurements transmitted at the predetermined transmission frequency may be at least one of: raw measurements taken by the respective sensor, measurements taken by the respective sensor which have been digitized, and measurements taken by the respective sensor which have been digitized and processed.

In some examples, the at least one frequency comprises the transmission frequency and at least one of the measurement frequency and the digitizing frequency, the transmission frequency being the frequency with which at least one of the following is transmitted: the measurements taken by the respective sensor at the measurement frequency, the digitized measurements which have been digitized at the digitizing frequency, and the processed measurements which have been processed at the predetermined processing frequency. As a result, the method may allow for adjusting frequencies involved in both the acquisition of measurements (e.g., at least one of the taking of measurements and the digitization of measurements) and the transmission of the acquired measurements. In this way, the method may allow for optimizing an adjustment of several frequencies affecting the same measurements, so that the method particularly allows enhancing a tradeoff among high accuracy of the motion measuring and/or of estimations based on measurements of the respective sensor (e.g., an estimation of temperature and/or respiration rate and/or pulse rate), low power consumption by the motion measuring system, and low congestion of a channel through which the measurements taken by the respective sensor are transmitted to the computing device.

The one or more optical sensors may comprise light sensors (e.g., photoelectric cells). For example, the one or more optical sensors comprise at least one of: an image sensor comprising light sensors for measuring visible light, an image sensor comprising light sensors for measuring infrared light, and a set of light sensors for detecting laser light. In some examples, the motion measuring system comprises a camera, the camera comprising the image sensor comprising light sensors for measuring visible light. In some examples, the motion measuring system comprises a camera, the camera comprising the image sensor comprising light sensors for measuring infrared light. In some examples the motion measuring system comprises a lidar, the lidar comprising the set of light sensors for detecting laser light, the laser light being emitted by the lidar and being used for measuring a distance to the one or more body members.

In some examples, the respective sensor is a sensor of the one or more optical sensors. Thereby, the method may allow for increasing and decreasing the at least one frequency for enhancing a tradeoff among high accuracy of the motion measuring, low power consumption by the motion measuring system and, if the transmission frequency is adjusted, low congestion of a channel through which the measurements taken by the respective optical sensor are transmitted to the computing device.

In some examples, a first adjustment value of the first plurality of adjustment values is associated with a first predetermined movement and a second adjustment value of the first plurality of adjustment values is associated with a second predetermined movement. The first predetermined movement causes a higher level of motion of the one or more body members than the second predetermined movement. An application of merely the first adjustment value to the respective predetermined frequency associated with the respective optical sensor may cause a higher increase in the respective frequency than an application of merely the second adjustment value to the respective predetermined frequency associated with the respective optical sensor. Thereby, an increase in the respective frequency may be higher for a predetermined movement requiring a relatively high level of motion of the one or more body members than an increase in the respective frequency for a predetermined movement requiring a relatively low level of motion of the one or more body members. In this way, the frequency may be adjusted to the level of motion of the one or more body members, enabling that accuracy of, for example, the motion measuring does not decrease, upon increasing the level of motion of the one or more body members according to the predetermined movement, as much as if the respective frequency were not increased.

In some examples, a first adjustment value of the first plurality of adjustment values is associated with a first predetermined movement and a second adjustment value of the first plurality of adjustment values is associated with a second predetermined movement. The first predetermined movement defines movement of a relatively weak body member (e.g., a neck) and the second predetermined movement only defines movement of one or more body members being relatively strong (e.g. a leg). An application of merely the first adjustment value to the respective predetermined frequency associated with the respective optical sensor may cause a higher increase in the respective frequency than an application of merely the second adjustment value to the respective predetermined frequency associated with the respective optical sensor. Thereby, an increase in the respective frequency may be higher for a predetermined movement defining movement of a relatively weak body member than an increase in the respective frequency for a predetermined movement defining movement of only relatively strong one or more body members. In this way, motion of a weak body member may be measured with relatively high accuracy, allowing increasing an accuracy of detection of a motion likely to cause an injury of the weak body member.

In some examples, the at least one sensor comprises one or more additional sensors arrangeable on the user, such as a sensor configured to measure a vital sign of the user. The vital sign may include at least one of: temperature of a body member of the user where the sensor is arranged, respiration rate of the user, and pulse rate of the user.

Since some vital signs, for example, respiration rate of the user, pulse rate of the user, and temperature of the user, may depend on the predetermined movement performed by the user, it may be advantageous that the at least one frequency associated with the respective sensor is configured based on the predetermined movement to be performed by the user. For example, a first predetermined movement which causes a relatively high increase in the vital sign (e.g., a high increase in the respiration rate) may be associated with a first adjustment value being different from a second adjustment value associated with a second predetermined movement which causes a relatively low increase in the vital sign, the first and the second adjustment values being values of the first plurality of adjustment values, the first adjustment value being associated with the first predetermined movement and the second adjustment value being associated with the second predetermined movement. An application of merely the first adjustment value to the respective predetermined frequency may cause a higher increase in the respective frequency than an application of merely the second adjustment value to the respective predetermined frequency.

Accuracy of measurements taken by sensors arranged on the user, such as temperature sensors configured to measure a temperature of the body member where the sensor is arranged, may decrease due to a movement of the sensor caused by the performance of the predetermined movement. This decreased accuracy of the measurements taken by the sensors may be at least partially compensated by increasing the at least one frequency, e.g., by taking measurements at a higher measurement frequency. Therefore, it may be desirable to configure the at least one frequency based on the predetermined movement to be performed by the user. For example, a first predetermined movement which causes a relatively high level of motion of the body member where the sensor is arranged may be associated with a first adjustment value being different from a second adjustment value associated with a second predetermined movement which causes a relatively low level of motion of the body member where the sensor is arranged, the first and the second adjustment values being values of the first plurality of adjustment values. An application of merely the first adjustment value to the respective predetermined frequency may cause a higher increase in the respective frequency than an application of merely the second adjustment value to the respective predetermined frequency.

Each adjustment value of the first plurality of adjustment values may be applied to the respective predetermined frequency by, for example, performing an operation (e.g., a sum, a subtraction, a multiplication, or a division) of the adjustment value and the respective predetermined frequency.

In some examples, the respective predetermined frequency of the measurement frequency associated with a sensor of the at least one sensor is the predetermined measurement frequency associated with the sensor. In other words, adjustment of the measurement frequency associated with the sensor may comprise applying an adjustment value of the first plurality of adjustment values to the predetermined measurement frequency associated with the sensor. In examples in which the at least one frequency to be configured comprises the measurement frequency associated with the respective sensor, the at least one predetermined frequency associated with the respective sensor comprises the predetermined measurement frequency associated with the respective sensor. The unit of the measurement frequency and of the predetermined measurement frequency may be: number of taken measurements per unit of time. For example, the respective sensor is an optical sensor of the one or more optical sensors and the unit of the measurement frequency and of the predetermined measurement frequency is the number of measurements of light taken per unit of time by the optical sensor. Adjusting the measurement frequency may allow for enhancing a tradeoff between low power consumption by the respective sensor and taking measurements at a frequency which is suitable for achieving a certain accuracy of the motion measuring and/or of estimations based on measurements of the respective sensor.

In some examples, the respective predetermined frequency of the digitizing frequency associated with a sensor of the at least one sensor is the predetermined digitizing frequency associated with the sensor. In other words, adjustment of the digitizing frequency associated with the sensor may comprise applying an adjustment value of the first plurality of adjustment values to the predetermined digitizing frequency associated with the sensor. In examples in which the at least one frequency to be configured comprises the digitizing frequency associated with the respective sensor, the at least one predetermined frequency associated with the respective sensor comprises the predetermined digitizing frequency associated with the respective sensor. The unit of the digitizing frequency and of the predetermined digitizing frequency may be: number of measurements digitized per unit of time, the unit of time being, for example, seconds. For example, the respective sensor is an optical sensor of the one or more optical sensors and the unit of the digitizing frequency and of the predetermined digitizing frequency is, for example, number of light measurements digitized per unit of time. Adjusting the digitizing frequency may allow for enhancing a tradeoff between low power consumption by the motion measuring system and providing digitized measurements at a frequency which is suitable for achieving a certain accuracy of the motion measuring and/or of estimations based on measurements of the respective sensor.

In some examples, the measurements digitized at the digitizing frequency are the measurements taken by the respective sensor at the measurement frequency. For example, the respective sensor is an optical sensor of the one or more optical sensors and may provide light measurements at a measurement frequency of 60 MHz, for example, by providing, every 100 milliseconds, six million voltages proportional to light intensity received by six million light sensors of the optical sensor, the voltages being digitized at 60 MHz. The measurement frequency of 60 MHz may be obtained with a camera capturing an image every 100 milliseconds, the camera measuring light with 6 million light sensors in the capture of each image.

In some examples, the measurements digitized at the digitizing frequency are the measurements taken by the respective sensor, the measurements being continuously taken over time. For example, the respective sensor may be configured to measure a physical magnitude in a continuous manner over time and the resulting measurements in some particular instants of time may be digitized at the digitizing frequency.

In some examples, the respective predetermined frequency of the processing frequency associated with a sensor of the at least one sensor is the predetermined processing frequency associated with the sensor. In other words, adjustment of the processing frequency associated with the sensor may comprise applying an adjustment value of the first plurality of adjustment values to the predetermined processing frequency associated with the sensor. In examples in which the at least one frequency to be configured comprises the processing frequency associated with the respective sensor, the at least one predetermined frequency associated with the respective sensor comprises the predetermined processing frequency associated with the respective sensor. The unit of the processing frequency and of the predetermined processing frequency may be: number of measurements processed per unit of time, the unit of time being, for example, seconds. Adjusting the processing frequency allows enhancing a tradeoff between low power consumption by the motion measuring system and providing processed measurements at a frequency which is suitable for achieving a certain accuracy of the motion measuring and/or of estimations based on measurements of the respective sensor.

In some examples, the respective predetermined frequency of the transmission frequency associated with a sensor of the at least one sensor is the predetermined transmission frequency associated with the sensor. In other words, adjustment of the transmission frequency associated with the sensor may comprise applying an adjustment value of the first plurality of adjustment values to the predetermined transmission frequency associated with the sensor. In examples in which the at least one frequency to be configured comprises the transmission frequency associated with the respective sensor, the at least one predetermined frequency associated with the respective sensor comprises the predetermined transmission frequency associated with the respective sensor. The unit of the transmission frequency and of the predetermined transmission frequency may be: number of transmitted measurements per unit of time, the unit of time being, for example, seconds. Adjusting the transmission frequency may allow for enhancing a tradeoff among transmitting measurements at a frequency which is suitable for achieving a certain accuracy of the motion measuring and/or of estimations based on measurements of the respective sensor, low power consumption by the motion measuring system, and low congestion of a channel through which the measurements are transmitted to the computing device.

In some examples, the measurements transmitted at the transmission frequency are digital measurements which have been processed. Thereby, the measurements received by the computing device may have already been preprocessed, allowing decreasing the processing load of the computing device.

In some examples, at least one of the measurement frequency associated with the respective sensor and the transmission frequency associated with the respective sensor is adjusted. Decreasing at least one of the measurement frequency and the transmission frequency may enable a relatively high reduction of power consumed by the motion measuring system when compared to decreasing the processing frequency and the digitizing frequency. This difference of power consumption decrease may be particularly high in motion measuring systems consuming more power in at least one of measuring at the measurement frequency and transmitting at the transmission frequency than in digitizing at the digitizing frequency and processing at the processing frequency.

In some examples, the digitizing frequency associated with the respective sensor and at least one of the measurement frequency associated with the respective sensor and the transmission frequency associated with the respective sensor are adjusted. Decreasing the digitizing frequency and decreasing at least one of the measurement frequency and the transmission frequency associated with a sensor may potentially enable a relatively high reduction of power consumed by the motion measuring system when compared to decreasing the processing frequency. This difference of power consumption decrease may be particularly high in motion measuring systems consuming more power in digitizing at the digitizing frequency and at least one of measuring at the measurement frequency and transmitting at the transmission frequency than in processing at the processing frequency.

In some examples, the measurement frequency associated with the respective sensor is equal to the transmission frequency associated with the respective sensor. Thereby, measurements may be taken and transmitted at the same frequency, so that the transmission frequency has a minimum value allowing transmitting all measurements taken by the respective sensor.

In some examples, the digitizing frequency associated with the respective sensor is equal to the transmission frequency associated with the respective sensor. Thereby, measurements may be digitized and transmitted at the same frequency, so that the transmission frequency has a minimum value allowing transmitting all measurements of the respective sensor digitized at the digitizing frequency or, in other words, so that the digitizing frequency has a minimum value allowing digitizing all measurements subsequently transmitted at the transmission frequency. Increasing just one of the frequencies, e.g., the digitizing frequency or the transmission frequency without increasing the other frequency, may increase power consumption by the motion measuring system without enabling an increase in accuracy of the motion measuring and/or estimations performed by the motion measuring system based on measurements of the respective sensor. For example, if the digitizing frequency is increased without increasing the transmission frequency, additionally digitized measurements resulting from the increase in the digitizing frequency would not be transmitted and hence would not be used by the motion measuring system for providing motion measuring and/or the estimations.

In some examples, the processing frequency associated with the respective sensor is equal to the transmission frequency associated with the respective sensor. Thereby, measurements may be processed and transmitted at the same frequency, so that the transmission frequency has a minimum value allowing transmitting of all measurements processed at the processing frequency or, in other words, so that the processing frequency has a minimum value allowing processing of all measurements subsequently transmitted at the transmission frequency.

In some examples, at least one of the following conditions apply: the transmission frequency is lower than or equal to the processing frequency, the processing frequency is lower than or equal to the digitizing frequency, and the digitizing frequency is lower than or equal to the measurement frequency. Thereby, the method may potentially enable the motion measuring system not to waste energy (or to waste less energy) in increasing frequencies which do not contribute to increasing accuracy of motion measuring and/or of estimations performed by the motion measuring system (e.g., prevents from increasing the transmission frequency above the digitizing frequency, thereby avoiding a transmission frequency being higher than required for transmission of all measurements digitized at the digitizing frequency).

In some examples, at least one of the following conditions apply: the transmission frequency is higher than the processing frequency, the processing frequency is higher than the digitizing frequency, and the digitizing frequency is higher than the measurement frequency. These examples may use up sampling wherein, for example, the processing frequency is higher than the measurement frequency and/or the digitizing frequency.

In some examples, the computing device of the motion measuring system is a first computing device and the motion measuring system comprises a second computing device.

In some examples, the second computing device comprises the data indicative of the first plurality of adjustment values.

In some examples, the second computing device is configured to determine an adjustment value of the first plurality of adjustment values based on the data indicative of the first plurality of adjustment values and data indicative of the predetermined movement to be performed by the user, the adjustment value being configured to be applied to the respective predetermined frequency associated with the sensor.

In some examples, the first computing device is configured to provide a sequence of motion measurement of the one or more body members based on measurements received from the one or more optical sensors.

In some examples, the first computing device comprises the data indicative of the first plurality of adjustment values.

In some examples, the first computing device is configured to determine an adjustment value of the first plurality of adjustment values based on the data indicative of the first plurality of adjustment values and data indicative of the predetermined movement to be performed by the user, the adjustment value being configured to be applied to the respective predetermined frequency associated with the sensor.

In some examples, each adjustment value of the first plurality of adjustment values is further associated with the user and a parameter indicative of a level of motion of the one or more body members as measured based on measurements of light of the one or more optical sensors in a previous performance of the predetermined movement. Each adjustment value of the first plurality of adjustment values may be associated with the user and with characteristics of a previous performance, by the user, of the predetermined movement. In this way, the one or more frequencies may be adjusted to each particular user, such as a user with a particular injury or with a particular disability.

Depending on the physical condition of the user, the execution of the predetermined movement may be faster or slower. For example, a person that suffers a lot of pain when executing the predetermined movement will probably execute the movement at a lower level of motion, e.g., with lower intensity, for example, slower and/or with lower accelerations and/or decelerations. Therefore, the association of each adjustment value of the first plurality of adjustment values with one user may enable the first plurality of adjustment values to take into account the physical condition of the user.

In the context of the present disclosure, the expression “level of motion” referred to one or more body members, such as “level of motion of one or more body members”, “level of motion of the one or more body members”, “level of motion of a body member”, or “level of motion of the body member”, refers to characteristics of a motion of the one or more body members, for example, to at least one of: a position of the one or more body members, an orientation of the one or more body members, and a relation of a position and/or orientation of the one or more body members with respect to time. The relation of a position and/or orientation of the one or more body members with respect to time comprises, among other possible units and types of measurements, at least one of: speed of the one or more body members, linear speed of the one or more body members, angular speed of the one or more body members, acceleration of the one or more body members, linear acceleration of the one or more body members, angular acceleration of the one or more body members, variation of acceleration of the one or more body members, variation of linear acceleration of the one or more body members, and variation of angular acceleration of the one or more body members. In some examples, the level of motion may be a level of motion defined by a motion of the one or more body members in an interval of time, the interval having a lower limit “a” and an upper limit “b” wherein a<b.

In some examples, the step of configuring at least based on the predetermined movement to be performed by the user is conducted either prior to the user starting to perform the predetermined movement, or while the user performs the predetermined movement. For example, the motion measuring system may receive or generate a signal indicative of the predetermined movement to be performed by the user, such as knee-ups, push-ups, or squats. In some examples, the signal may be generated by a selection, e.g., by the user, of the predetermined movement via a user interface of the motion measuring system. In some examples, the signal may be generated based on data indicative of movements to be performed by the user and associated with the user. The motion measuring system may adjust the at least one frequency before the user starts performing the predetermined movement or while the user performs the predetermined movement. In some examples, the step of configuring at least based on the predetermined movement to be performed by the user is conducted after the previous performance, by the user, of the predetermined movement and before another performance, by the user, of the predetermined movement.

In some examples, the method further comprises configuring the at least one frequency associated with the respective sensor at least based on measurements of light of the one or more optical sensors, thereby modifying a value of the at least one predetermined frequency associated with the respective sensor. The motion measuring system may comprise data indicative of a second plurality of adjustment values for configuring the respective at least one frequency based on measurements of the one or more optical sensors, each adjustment value of the second plurality of adjustment values being associated with a level of motion of the one or more body members as measured based on measurements of light of the one or more optical sensors, the level of motion being within a predetermined motion range, and each adjustment value of the second plurality of adjustment values being configured to be applied to the respective predetermined frequency associated with the sensor.

The adjustment of the one or more frequencies may comprise applying, to the respective predetermined frequency, an adjustment value of the first plurality of adjustment values and an adjustment value of the second plurality of adjustment values. The application of the adjustment value of the second plurality of adjustment values may allow for adjusting the one or more frequencies to characteristics of the actual movements performed by the user. For example, a user may perform the predetermined movement in a way such that the one or more body members may be subjected to a higher level of motion (e.g., faster) than it is expected according to the adjustment value of the first plurality of adjustment values associated with the predetermined movement. The adjustment value of the second plurality of adjustment values associated with the higher level of motion of the one or more body members of the user may be a first adjustment value such that an application of merely the first adjustment value and the adjustment value of the first plurality of adjustment values to the respective at least one predetermined frequency results in the respective at least one frequency having a higher value than a value of the respective at least one frequency resulting from an application of merely the adjustment value of the first plurality of adjustment values to the respective at least one predetermined frequency. In some examples, the method thus allows increasing the at least one frequency for allowing increasing the accuracy of the motion measuring and/or of estimations based on measurements of the respective sensor at the cost of increasing power consumption by the motion measuring system.

The level of motion may be within the predetermined motion range. The level of motion comprises, for example, estimated positions and/or speeds and/or accelerations of the one or more body members based on measurements taken by the one or more optical sensors.

In a second aspect of the disclosure, a method for adjusting one or more frequencies associated with at least one sensor of a motion measuring system is provided. The at least one sensor comprises one or more optical sensors, and the one or more optical sensors are configured to be arranged at a distance from a user so that the one or more optical sensors receive light from one or more body members of the user. The one or more optical sensors may be configured to provide measurements of light received from the one or more body members to measure motion of the one or more body members. The at least one sensor may comprise one or more additional sensors, the one or more additional sensors being arrangeable on the user.

The method may comprise configuring at least one frequency associated with the respective sensor of the at least one sensor at least based on measurements of light of the one or more optical sensors, thereby modifying a value of at least one predetermined frequency associated with the respective sensor. The at least one predetermined frequency associated with the respective sensor may comprise at least one of: a predetermined measurement frequency with which the respective sensor measures a respective physical magnitude, a predetermined digitizing frequency with which measurements taken by the respective sensor are digitized, a predetermined processing frequency with which the digitized measurements are processed, and a predetermined transmission frequency with which measurements taken by the respective sensor are transmitted to a computing device. The computing device may be configured to receive measurements from the respective sensor.

The at least one frequency to be configured may comprise at least one of: a measurement frequency, a digitizing frequency, a processing frequency, and a transmission frequency.

The motion measuring system may comprise data indicative of a second plurality of adjustment values for configuring the respective at least one frequency based on measurements of the one or more optical sensors, each adjustment value of the second plurality of adjustment values being associated with a level of motion of the one or more body members as measured based on measurements of light of the one or more optical sensors, the level of motion being within a predetermined motion range, and each adjustment value of the second plurality of adjustment values being configured to be applied to the respective predetermined frequency associated with the sensor.

Accordingly, at least one of the measurement frequency, the digitizing frequency, the processing frequency, and the transmission frequency associated with the sensor may be configured based on a second plurality of adjustment values wherein each adjustment value of the second plurality of adjustment values is associated with a level of motion of the one or more body members as measured based on measurements of light of the one or more optical sensors, the level of motion being within a predetermined motion range.

Similarly to the first aspect of the disclosure, the second aspect of the disclosure may allow for increasing and decreasing the at least one frequency for enhancing a tradeoff among high accuracy of the motion measuring and/or of estimations based on measurements of the respective sensor (e.g., an estimation of at least one of: temperature, respiration rate, and pulse rate), low power consumption by the motion measuring system and, if the transmission frequency is adjusted, low congestion of a channel through which the measurements taken by the respective sensor are transmitted to the computing device. In addition, the second aspect of the disclosure may allow for detecting dynamic movements, such as running and jumping.

In some examples, the use of the second plurality of adjustment values does not require, but is compatible with, the use of the first plurality of adjustment values.

The level of motion may comprise, for example, estimated positions and/or speeds and/or accelerations of the one or more body members based on measurements of light of the one or more optical sensors.

The measurements transmitted at the predetermined transmission frequency may be at least one of: raw measurements taken by the respective sensor, measurements taken by the respective sensor which have been digitized, and measurements taken by the respective sensor which have been digitized and processed.

In some examples, the at least one frequency comprises the transmission frequency and at least one of the measurement frequency and the digitizing frequency. The transmission frequency may be the frequency with which the respective sensor transmits at least one of: the measurements taken by the respective sensor at the measurement frequency, the digitized measurements which have been digitized at the digitizing frequency, and the processed measurements which have been processed at the predetermined processing frequency. Thereby, the method may allow for adjusting frequencies involved in both the acquisition of measurements (e.g., at least one of the taking of measurements and the digitization of measurements) and the transmission of the acquired measurements. In this way, the method may allow for optimizing an adjustment of several frequencies affecting the same measurements, so that the method particularly allows enhancing a tradeoff among high accuracy of the motion measuring and/or of estimations based on measurements of the respective sensor, low power consumption by the motion measuring system and low congestion of a channel through which the measurements taken by the respective sensor are transmitted to the computing device.

The one or more optical sensors may comprise light sensors (e.g., photoelectric cells). For example, the one or more optical sensors comprise at least one of: an image sensor comprising light sensors for measuring visible light, an image sensor comprising light sensors for measuring infrared light, and a set of light sensors for detecting laser light. In some examples, the motion measuring system comprises a camera, the camera comprising the image sensor comprising light sensors for measuring visible light. In some examples, the motion measuring system comprises a camera, the camera comprising the image sensor comprising light sensors for measuring infrared light. In some examples, the motion measuring system comprises a lidar, the lidar comprising the set of light sensors for detecting laser light, the laser light being emitted by the lidar and being used for measuring a distance to the one or more body members.

In some examples, the respective sensor is a sensor of the one or more optical sensors. Thereby, the method may allow for increasing and decreasing the at least one frequency for enhancing a tradeoff among high accuracy of the motion measuring, low power consumption by the motion measuring system and, if the transmission frequency is adjusted, low congestion of a channel through which the measurements taken by the respective optical sensor are transmitted to the computing device.

In some examples, an acquisition frequency is adjusted. The acquisition frequency may be a frequency with which the camera captures images, e.g., the number of images captured by the camera per unit of time. The acquisition frequency may be dependent on the measurement frequency and on the digitizing frequency associated with the one or more optical sensors of the camera. In a capture of an image light is measured by light sensors of the one or more optical sensors of the camera and the measurements of light are digitized into digital data defining the captured image. Since the capture of each image consumes power and provides data about a motion performed by the user, adjusting the acquisition frequency may allow for enhancing a tradeoff among acquiring images at a frequency which is suitable for achieving a certain accuracy of the motion measuring and low power consumption by the motion measuring system.

In some examples, a first adjustment value of the second plurality of adjustment values is associated with a first level of motion of the one or more body members and a second adjustment value of the second plurality of adjustment values is associated with a second level of motion of the one or more body members, the first level of motion being higher than the second level of motion. An application of merely the first adjustment value to the respective predetermined frequency associated with the respective optical sensor may cause a higher increase in the respective frequency than an application of merely the second adjustment value to the respective predetermined frequency associated with the respective optical sensor. Thereby, the increase in the respective frequency may be higher for a relatively high level of motion than the increase in the respective frequency for a relatively low level of motion. In this way, the frequency may be adjusted to the level of motion of the one or more body members, enabling that accuracy of, for example, the motion measuring of the one or more body members does not decrease, upon increasing the level of motion of the one or more body members, as much as if the respective frequency were not increased.

In some examples, the at least one sensor comprises one or more additional sensors arrangeable on the user, for example, a sensor configured to measure a vital sign of the user. The vital sign may be at least one of: temperature of a body member of the user where the sensor is arranged, respiration rate of the user, and pulse rate of the user.

Since some vital signs, for example, respiration rate of the user, pulse rate of the user and temperature of the user depend on the level of motion of the one or more body members, it may be advantageous that the at least one frequency associated with the respective sensor is configured based on the level of motion of the one or more body members. For example, a high level of motion which causes a relatively high increase in the vital sign may be associated with a first adjustment value being different from a second adjustment value associated with a low level of motion which causes a relatively lower increase in the vital sign, the first and the second adjustment values being values of the second plurality of adjustment values. An application of merely the first adjustment value to the respective predetermined frequency may cause a higher increase in the respective frequency than an application of merely the second adjustment value to the respective predetermined frequency.

Accuracy of measurements taken by sensors arranged on the user, such as temperature sensors configured to measure a temperature of the body member where the sensor is arranged, may decrease due to a movement of the sensor caused by a movement of the one or more body members. This decreased accuracy of the measurements taken by the sensors may be at least partially compensated by increasing the at least one frequency, e.g., by taking measurements at a higher measurement frequency. Therefore, it may be desirable to configure the at least one frequency based on a level of motion of the one or more body members and, more particularly, based on a level of motion of the body member where the sensor is arranged. For example, a first adjustment value of the second plurality of adjustment values is associated with a first level of motion of the one or more body members and a second adjustment value of the second plurality of adjustment values is associated with a second level of motion of the one or more body members, the first level of motion being higher than the second level of motion. An application of merely the first adjustment value to the respective predetermined frequency associated with the respective sensor may cause a higher increase in the respective frequency than an application of merely the second adjustment value to the respective predetermined frequency associated with the respective sensor.

In some examples, the computing device of the motion measuring system is a first computing device and the motion measuring system comprises a second computing device.

In some examples, the second computing device comprises the data indicative of the second plurality of adjustment values.

In some examples, the second computing device is configured to determine an adjustment value of the second plurality of adjustment values based on the data indicative of the second plurality of adjustment values and data indicative of a level of motion of the one or more body members as measured based on measurements of light of the one or more optical sensors, the level of motion being within a predetermined motion range.

In some examples, the first computing device is configured to provide a sequence of motion measurement of the one or more body members based on measurements received from the one or more optical sensors.

In some examples, the first computing device comprises the data indicative of the second plurality of adjustment values.

In some examples, the first computing device is configured to determine an adjustment value of the second plurality of adjustment values based on the data indicative of the second plurality of adjustment values and data indicative of a level of motion the one or more body members as measured based on measurements of light of the one or more optical sensors, the level of motion being within a predetermined motion range.

In some examples, a first adjustment value of the second plurality of adjustment values is associated with a first level of motion of the one or more body members, and a second adjustment value of the second plurality of adjustment values is associated with a second level of motion of the one or more body members. The first level of motion may be higher than the second level of motion. The first adjustment value may be such that the application of merely the first adjustment value to the respective predetermined frequency associated with the sensor provides a higher frequency than a frequency provided by the application of merely the second adjustment value to the respective predetermined frequency associated with the sensor.

Thereby, the method may allow for increasing accuracy of motion measuring and/or of estimations based on measurements of the respective sensor upon an increased level of motion compared to not increasing the respective frequency upon increasing the level of motion.

The adjustment value of the second plurality of adjustment values may be applied to the respective predetermined frequency by, for example, performing an operation (e.g., a sum, a subtraction, a multiplication, or a division) of the adjustment value and the respective predetermined frequency.

In some examples, the respective predetermined frequency of the measurement frequency associated with a sensor of the at least one sensor is the predetermined measurement frequency associated with the sensor. In other words, adjustment of the measurement frequency associated with the sensor may comprise applying an adjustment value of the second plurality of adjustment values to the predetermined measurement frequency associated with the sensor. In examples in which the at least one frequency to be configured comprises the measurement frequency associated with the respective sensor, the at least one predetermined frequency associated with the respective sensor comprises the predetermined measurement frequency associated with the respective sensor. The unit of the measurement frequency and of the predetermined measurement frequency may be: number of taken measurements per unit of time. For example, the respective sensor is an optical sensor of the one or more optical sensors and the unit of the measurement frequency and of the predetermined measurement frequency is, for example, number of measurements of light taken per unit of time by the optical sensor. Adjusting the measurement frequency may allow for enhancing a tradeoff between low power consumption by the respective sensor and taking measurements at a frequency which is suitable for achieving a certain accuracy of the motion measuring and/or of estimations based on measurements of the respective sensor.

In some examples, the respective predetermined frequency of the digitizing frequency associated with a sensor of the at least one sensor is the predetermined digitizing frequency associated with the sensor. In other words, adjustment of the digitizing frequency associated with the sensor may comprise applying an adjustment value of the second plurality of adjustment values to the predetermined digitizing frequency associated with the sensor. In examples in which the at least one frequency to be configured comprises the digitizing frequency associated with the respective sensor, the at least one predetermined frequency associated with the respective sensor comprises the predetermined digitizing frequency associated with the respective sensor. The unit of the digitizing frequency and of the predetermined digitizing frequency may be: number of measurements digitized per unit of time, the unit of time being, for example, seconds. For example, the respective sensor is an optical sensor of the one or more optical sensors and the unit of the digitizing frequency and of the predetermined digitizing frequency is, for example, number of light measurements digitized per unit of time. Adjusting the digitizing frequency may allow for enhancing a tradeoff between low power consumption by the motion measuring system and providing digitized measurements at a frequency which is suitable for achieving a certain accuracy of the motion measuring and/or of estimations based on measurements of the respective sensor.

In some examples, the measurements digitized at the digitizing frequency are the measurements taken by the respective sensor at the measurement frequency. For example, the respective sensor is an optical sensor of the one or more optical sensors and may provide light measurements at a measurement frequency of 60 MHz, for example, by providing, every 100 milliseconds, six million voltages proportional to light intensity received by six million light sensors of an optical sensor, the voltages being digitized at 60 MHz. The measurement frequency of 60 MHz may be obtained with a camera capturing an image every 100 milliseconds, the camera measuring light with 6 million light sensors in the capture of each image.

In some examples, the measurements digitized at the digitizing frequency are measurements taken by the respective sensor, the measurements being continuously taken over time. For example, the respective sensor may be configured to measure a physical magnitude in a continuous manner over time and the resulting measurements in some particular instants of time may be digitized at the digitizing frequency.

In some examples, the respective predetermined frequency of the processing frequency associated with a sensor of the at least one sensor is the predetermined processing frequency associated with the sensor. In other words, adjustment of the processing frequency associated with the sensor may comprise applying an adjustment value of the second plurality of adjustment values to the predetermined processing frequency associated with the sensor. In examples in which the at least one frequency to be configured comprises the processing frequency associated with the respective sensor, the at least one predetermined frequency associated with the respective sensor comprises the predetermined processing frequency associated with the respective sensor. The unit of the processing frequency and of the predetermined processing frequency may be: number of measurements processed per unit of time, the unit of time being, for example, seconds. Adjusting the processing frequency may allow for enhancing a tradeoff between low power consumption by the motion measuring system and providing processed measurements at a frequency which is suitable for achieving a certain accuracy of the motion measuring and/or of estimations based on measurements of the respective sensor.

In some examples, the respective predetermined frequency of the transmission frequency associated with a sensor of the at least one sensor is the predetermined transmission frequency associated with the sensor. In other words, adjustment of the transmission frequency associated with the sensor may comprise applying an adjustment value of the second plurality of adjustment values to the predetermined transmission frequency associated with the sensor. In examples in which the at least one frequency to be configured comprises the transmission frequency associated with the respective sensor, the at least one predetermined frequency associated with the respective sensor comprises the predetermined transmission frequency associated with the respective sensor. The unit of the transmission frequency and of the predetermined transmission frequency may be: number of transmitted measurements per unit of time, the unit of time being, for example, seconds. Adjusting the transmission frequency may allow for enhancing a tradeoff among transmitting measurements at a frequency which is suitable for achieving a certain accuracy of the motion measuring and/or of estimations based on measurements of the respective sensor, low power consumption by the motion measuring system, and low congestion of a channel through which the measurements are transmitted to the computing device.

In some examples, at least one of the measurement frequency associated with the respective sensor and the transmission frequency associated with the respective sensor is adjusted. Decreasing at least one of the measurement frequency and the transmission frequency may enable a relatively high reduction of power consumed by the motion measuring system compared to decreasing the processing frequency and the digitizing frequency. This difference of power consumption decrease may be particularly high in motion measuring systems consuming more power in at least one of measuring at the measurement frequency and transmitting at the transmission frequency than in digitizing at the digitizing frequency and processing at the processing frequency.

In some examples, the digitizing frequency associated with the respective sensor and at least one of the measurement frequency associated with the respective sensor and the transmission frequency associated with the respective sensor are adjusted. Decreasing the digitizing frequency and decreasing at least one of the measurement frequency and the transmission frequency associated with a sensor may enable a relatively high reduction of power consumed by the motion measuring system compared to decreasing the processing frequency. This difference of power consumption decrease may be particularly high in motion measuring systems consuming more power in digitizing at the digitizing frequency and at least one of measuring at the measurement frequency and transmitting at the transmission frequency than in processing at the processing frequency.

In some examples, the measurement frequency associated with the respective sensor is equal to the transmission frequency associated with the respective sensor. Thereby, measurements may be taken and transmitted at the same frequency, so that the transmission frequency has a minimum value allowing transmitting all measurements taken by the respective sensor.

In some examples, the digitizing frequency associated with the respective sensor is equal to the transmission frequency associated with the respective sensor. Thereby, measurements may be digitized and transmitted at the same frequency, so that the transmission frequency has a minimum value allowing transmitting all measurements digitized at the digitizing frequency or, in other words, so that the digitizing frequency has a minimum value allowing digitizing all measurements subsequently transmitted at the transmission frequency. Increasing just one of the frequencies, e.g., the digitizing frequency or the transmission frequency without increasing the other frequency may increase power consumption by the motion measuring system without enabling an increase in accuracy of the motion measuring and/or the estimations performed by the motion measuring system based on measurements of the respective sensor. For example, if the digitizing frequency is increased without increasing the transmission frequency, additionally digitized measurements resulting from the increase in the digitizing frequency would not be transmitted and hence would not be used by the motion measuring system for providing a motion measuring sequence and/or the estimations.

In some examples, the processing frequency associated with the respective sensor is equal to the transmission frequency associated with the respective sensor. Thereby, measurements may be processed and transmitted at the same frequency, so that the transmission frequency has a minimum value allowing transmitting all measurements processed at the processing frequency or, in other words, so that the processing frequency has a minimum value allowing processing of all measurements subsequently transmitted at the transmission frequency.

In some examples, at least one of the following conditions apply: the transmission frequency is lower than or equal to the processing frequency, the processing frequency is lower than or equal to the digitizing frequency, and the digitizing frequency is lower than or equal to the measurement frequency. Thereby, the method may potentially enable the motion measuring system not to waste energy (or to waste less energy) in increasing frequencies which do not contribute to increasing accuracy of motion measuring and/or of estimations performed based on measurements of the respective sensor (e.g., prevents from increasing the transmission frequency above the digitizing frequency, thereby avoiding a transmission frequency being higher than required for transmission of all measurements digitized at the digitizing frequency).

In some examples, at least one of the following conditions apply: the transmission frequency is higher than the processing frequency, the processing frequency is higher than the digitizing frequency, and the digitizing frequency is higher than the measurement frequency. These examples may use upsampling wherein, for example, the processing frequency is higher than the measurement frequency and/or the digitizing frequency.

In some examples, the step of configuring based on the measurements comprises configuring the transmission frequency associated with the respective sensor with an adjustment value of the second plurality of adjustment values corresponding to no transmission when the level of motion being within the predetermined motion range is within a predetermined motion range associated with the one or more body members being motionless.

This may enable the motion measuring system to reduce or minimize energy wasted on transmission of measurements when the one or more body members are motionless or substantially motionless and decrease congestion of a channel through which the measurements of the respective sensor are transmitted. The transmission frequency, e.g., the frequency with which the measurements of the respective sensor are transmitted to the computing device of the motion measuring system, may be set to zero. The level of motion comprises, for example, estimated positions and/or speeds and/or accelerations of the one or more body members, the estimations being based on measurements of light of the one or more optical sensors. The predetermined range associated with being motionless is, for example, a range of speed or acceleration which comprises the value of speed equal to zero or acceleration equal to zero. The predetermined range associated with being motionless may be based on the body member where the respective sensor is arranged or must be arranged. The predetermined range associated with being motionless for a first body member may be different to the predetermined range associated with a body member which is not the first body member. For example, the predetermined range associated with a motionless toe may be narrower than the predetermined range associated with a motionless lower leg.

In some examples, the method further comprises: transmitting, to the computing device, data indicative of at least temporal no transmission of measurements from the respective sensor having an associated transmission frequency configured with the adjustment value corresponding to no transmission; and/or after non reception by the computing device of measurements from the respective sensor during a predetermined period of time if the data indicative of at least temporal no transmission has not been received, processing, by at least one processor, of most recent measurements received from the one or more optical sensors to determine whether a level of motion of the one or more body members is within the predetermined motion range associated with being motionless, the motion measuring system halting a motion measuring procedure otherwise.

In this way, the method may allow for detecting the level of motion of the one of more body members, the level of motion being within the predetermined motion range associated with being motionless. In some examples, if the transmission frequency associated with the respective sensor has been configured with the adjustment value corresponding to no transmission, the data indicative of temporal no transmission of measurements from the respective sensor is transmitted to the computing device. In other words, the data indicative of an absence of transmission of measurements taken by the respective sensor is transmitted to the computing device, the absence of transmission of measurements being temporal.

In some examples, the method comprises configuring a measurement frequency associated with the one or more optical sensors with an adjustment value corresponding to measuring when a transmission frequency associated with the respective sensor has been configured with the adjustment value corresponding to no transmission. Thereby, the one or more optical sensors may keep taking measurements when the transmission frequency associated with the respective sensor has been configured with the adjustment value corresponding to no transmission, so that the motion measuring system may detect when the level of motion of the one or more body members is not within the predetermined motion range associated with the one or more body members being motionless.

In some examples, the method comprises transmitting, to the computing device, data indicative of transmission of measurements from the respective sensor when the level of motion is not within the predetermined motion range associated with being motionless and the respective sensor has an associated transmission frequency configured with the adjustment value corresponding to no transmission.

In some examples, if the computing device does not receive measurements from the respective sensor during a predetermined period of time, the data indicative of at least temporal no transmission has not been received and the most recent measurements received from the one or more optical sensors are associated with a level of motion of the one or more body members within the predetermined motion range associated with the one or more body members being motionless, then it may be considered that the one or more body members are motionless or substantially motionless.

In some examples, if the computing device does not receive measurements from the respective sensor during a predetermined period of time, the data indicative of at least temporal no transmission has not been received and the most recent measurements received from the one or more optical sensors are associated with a level of motion of the one or more body members not within the predetermined motion range associated with the one or more body members being motionless, then it may be considered that the respective sensor is not working properly and hence it may be appropriate to halt the motion measuring procedure. In such cases, the method may include providing an alert of a fault condition of the motion measuring system, e.g., to a remote monitoring device.

In some examples, the method further comprises configuring the at least one frequency associated with the respective sensor at least based on a predetermined movement to be performed by the user, thereby modifying a value of the at least one predetermined frequency associated with the respective sensor. The motion measuring system may comprise data indicative of a first plurality of adjustment values for configuring the respective at least one frequency based on a predetermined movement to be performed by the user, each adjustment value of the first plurality of adjustment values being associated with one predetermined movement to be performed by the user, and each adjustment value of the first plurality of adjustment values being configured to be applied to the respective predetermined frequency associated with the sensor. Thereby, the adjustment of the one or more frequencies may involve applying at least one value of the first plurality of adjustment values and at least one value of the second plurality of adjustment values to the respective predetermined frequency or the respective predetermined frequencies.

In some examples, each adjustment value of the first plurality of adjustment values is further associated with the user and a parameter indicative of a level of motion of the one or more body members as measured based on measurements of light of the one or more optical sensors in a previous performance of the predetermined movement.

In some examples, the step of configuring at least based on the predetermined movement to be performed by the user is conducted either prior to the user starting to perform the predetermined movement, or while the user performs the predetermined movement.

In some examples, the method further comprises at least one of: taking measurements by each of the at least one sensor, digitizing measurements taken by each of the at least one sensor, processing the digitized measurements, and transmitting, to the computing device, measurements taken by each of the at least one sensor at the respective frequency of the at least one frequency.

A third aspect of the disclosure provides a device or system comprising: means or components adapted to execute a method according to the first or the second aspect of the disclosure.

A fourth aspect of the disclosure provides a motion measuring system comprising: at least one sensor comprising one or more optical sensors and preferably comprising one or more additional sensors, at least one processor; at least one memory; and a computing device configured to receive measurements from a sensor of the at least one sensor. The at least one processor may be configured to conduct the method of the first or the second aspect of the disclosure.

In some examples, the computing device comprises the at least one processor. Thereby, the number of processing devices of the motion measuring system may be minimized.

A fifth aspect of the disclosure provides a computer program product that has instructions which, when executed by at least one processor, cause a motion measuring system to carry out the steps of a method according to the first or the second aspect of the disclosure.

Similar benefits as those described for the first and second aspects of the disclosure may also be applicable to the third and/or fourth and/or fifth aspects of the disclosure.

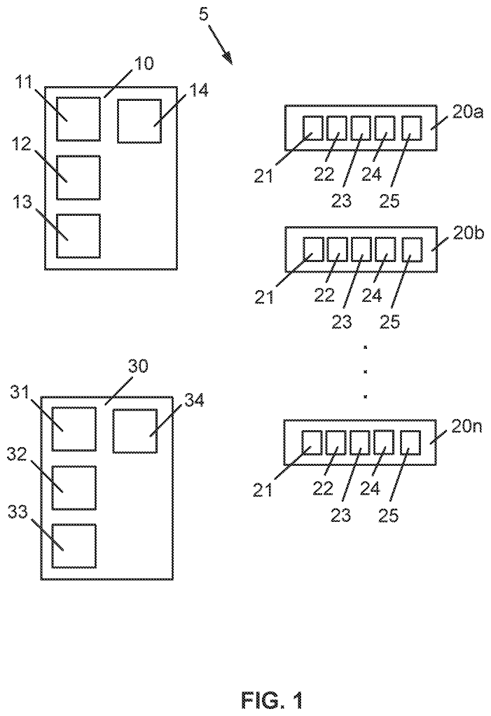

FIG. 1 diagrammatically shows a motion measuring system 5 in accordance with some examples. The motion measuring system 5 includes a computing device 10, which may, for example, be a tablet, a mobile phone, or a personal computer. The motion measuring system 5 further includes a camera 30 and one or more additional devices 20a-20n arrangeable on a user.

The camera 30 comprises an optical sensor 31. In FIG. 1, the optical sensor 31 is an image sensor comprising light sensors. The camera 30 includes at least one processor 32, at least one memory 33, and a wireless communications module 34 for transmitting radiofrequency signals to and receiving radiofrequency signals from the computing device 10. For example, the camera 30 transmits advertisement packages, data packets with identification data (e.g., one or more identities, keys, etc.), data packets with measurements of light of the light sensors of the optical sensor 31, data packets with directions computed by the camera 30, or combinations thereof, and receives packets from the computing device 10 with, for example, at least one of: a predetermined measurement frequency with which the optical sensor 31 of the camera 30 receiving the packets measures light, a predetermined digitizing frequency with which the camera 30 digitizes measurements taken by the optical sensor 31, a predetermined processing frequency with which the at least one processor 32 of the camera 30 processes the digitized measurements, a predetermined transmission frequency with which the camera 30 transmits measurements taken by the optical sensor 31 to the computing device 10, instructions to configure a measurement frequency and/or a digitizing frequency and/or a processing frequency and/or a transmission frequency associated with the optical sensor 31, data indicative of a first plurality of adjustment values for configuring at least one of the measurement frequency, the digitizing frequency, the processing frequency and the transmission frequency associated with the optical sensor 31, data indicative of a second plurality of adjustment values for configuring at least one of the measurement frequency, the digitizing frequency, the processing frequency and the transmission frequency associated with the optical sensor 31, data indicative of an adjustment value for configuring the measurement frequency and/or the digitizing frequency and/or the processing frequency and/or the transmission frequency associated with the optical sensor 31.

At least when no wireless communications connections are established with the computing device 10, the radiofrequency signals of the camera 30 include advertisement packages for indicating its presence and that the camera 30 is active. Once the wireless communications connections are established (using a technology and protocol known by a skilled person, for instance but without limitation, Bluetooth and Bluetooth Low Energy communications, cellular network communications, such as GSM, UMTS or LTE, wireless LAN communications, etc.) with the computing device 10, the radiofrequency signals of the camera 30 may include identification data and/or the measurements based on which the motion measuring sequence will be provided by the computing device 10.

The camera 30 is arrangeable at a distance from the body of a person so that the measurements provided by the optical sensor 31 can be processed by the computing device 10 to provide a motion measuring sequence of the person.

The one or more additional devices 20a-20n each include one or more additional sensors 21, at least one processor 22, at least one memory 23, and a second wireless communications module 24 for transmitting radiofrequency signals to and receiving radiofrequency signals from the computing device 10. For example, the one or more additional devices 20a-20n transmit advertisement packages, data packets with identification data (e.g. one or more identities or keys), data packets with measurements of the one or more additional sensors 21, data packets with directions computed by the one or more additional devices 20a-20n, or combinations thereof, and receive packets from the computing device 10 with, for example, at least one of: a predetermined measurement frequency with which the one or more additional sensors 21 of an additional device of the one or more additional devices 20a-20n receiving the packets measures a respective physical magnitude, a predetermined digitizing frequency with which the additional device receiving the packets digitizes measurements taken by the one or more additional sensors 21 of the additional device, a predetermined processing frequency with which the additional device receiving the packets processes the digitized measurements, a predetermined transmission frequency with which the additional device receiving the packets transmits measurements taken by the one or more additional sensors 21 to the computing device 10, instructions to configure a measurement frequency and/or a digitizing frequency and/or a processing frequency and/or a transmission frequency associated with the one or more additional sensors 21 of the additional device receiving the packets, data indicative of a first plurality of adjustment values for configuring at least one of the measurement frequency, the digitizing frequency, the processing frequency and the transmission frequency associated with the one or more additional sensors 21 of the additional device receiving the packets, data indicative of a second plurality of adjustment values for configuring at least one of the measurement frequency, the digitizing frequency, the processing frequency and the transmission frequency associated with the one or more additional sensors 21 of the additional device receiving the packets, data indicative of an adjustment value for configuring the measurement frequency and/or the digitizing frequency and/or the processing frequency and/or the transmission frequency associated with the one or more additional sensors 21 of the additional device receiving the packets. At least when no wireless communications connections are established with the computing device 10, the radiofrequency signals of the one or more additional devices 20a-20n include advertisement packages for indicating their presence and that they are active. Once the wireless communications connections are established (using a technology and protocol known by a skilled person, for instance but without limitation, Bluetooth and Bluetooth Low Energy communications, cellular network communications, such as GSM, UMTS or LTE, wireless LAN communications, etc.) with the computing device 10, the radiofrequency signals of the one or more additional devices 20a-20n may include identification data and/or the measurements based on which an estimation will be provided by the computing device 10.

Each of the one or more additional devices 20a-20n is adapted to be arranged on the body of a person so that the measurements provided by each of the one or more additional devices 20a-20n can be processed by the computing device 10 to provide an estimation of a vital sign of the person based on the measurements from the one or more additional sensors 21 of the one or more additional devices 20a-20n. The one or more additional devices 20a-20n may be attached to body members of the person by means of an attaching device 25 like, for instance, straps, Velcro (e.g., hook-and-loop fasteners), or other means, that each of the one or more additional devices 20a-20n itself comprises. The additional devices 20a-20n or their additional sensors 21 may thus be referred to as body member sensors.

Each additional device 20a-20n may be powered by one or more batteries, e.g., a rechargeable battery. The camera 30 may also be powered by one or more batteries, e.g., a rechargeable battery.

In some examples, the one or more additional sensors 21 comprise one or more vital sign sensors. The vital sign sensors may include at least one of: one or more temperature sensors for measuring a temperature of the user, one or more pulse rate sensors for measuring a pulse rate of the user, one or more respiration rate sensors for measuring a respiration rate of the user, or a combination of two or more thereof.

The computing device 10 may include at least one processor 11, at least one memory 12, and a third wireless communications module 13 for transmitting radiofrequency signals to the camera 30 and the one or more additional devices 20a-20n and receiving radiofrequency signals therefrom.

The motion measuring system 5 may also include at least one device 14 (which may be part of the computing device 10 or be separate from the computing device 10) for providing user perceptible signals, such as a screen, loudspeakers, or a combination thereof, to name a few examples. That is to say, the at least one device 14 may comprise one or more of visual output means (e.g. screen, LEDs), audio output means (e.g. loudspeakers), vibrating means (e.g. a vibrator), or other means for providing user perceptible signals in the form of sounds, vibration, animated graphics, etc.

When the at least one device 14 comprises a screen or other display, the computing device 10 is capable of showing instructions and/or information to the intended user about the operation of the motion measuring system 5 and the motion measuring procedure to be conducted with the system 5, for example predetermined movements that are to be performed by an intended user of the motion measuring system 5, results of the exercises performed by the user, etc. The device 14 may thus provide a user interface (UI) to present instructions and/or information to the user and/or to receive inputs from the user. The computing device 10 stores, in the at least one memory 12 data relative to the physical exercises, e.g., predetermined movements, of intended users. Any of these data can be transmitted to and/or received from another electronic device thanks to the third wireless communications module 13. For example, a therapist is able to receive the feedback at a computing device in a remote location, such as a hospital, so as to monitor the evolution of the person. Based on the feedback received, the therapist is able to adjust the difficulty of the movement(s), the number of repetitions thereof, prescribe new movements, etc. so that the person may further exercise using the motion measuring system 5.

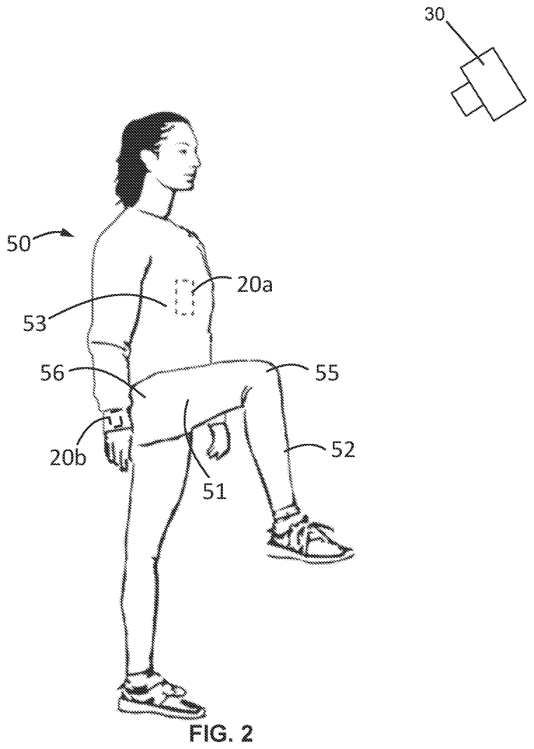

FIG. 2 shows a person 50 (also referred to as a user 50) performing a first predetermined movement while wearing additional devices 20a and 20b, according to some examples.

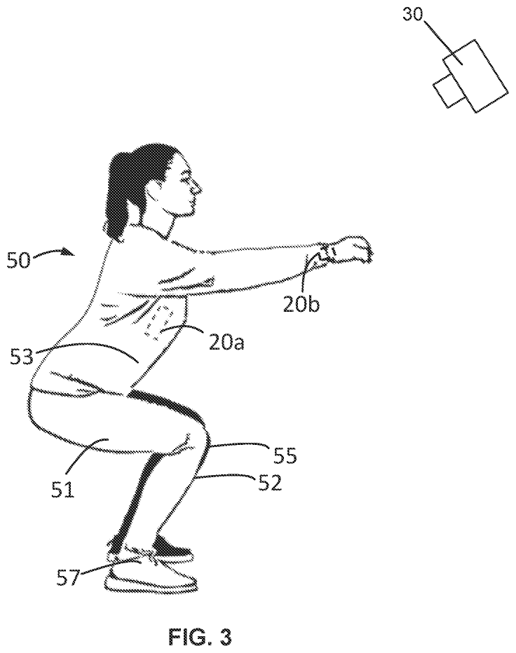

FIG. 3 shows the person 50 performing a second predetermined movement while wearing additional devices 20a and 20b, according to some examples. Both FIG. 2 and FIG. 3 also illustrate the camera 30 spaced apart from the person 50 and arranged such that the optical sensor 31 can capture the person 50 while doing the predetermined movements.

The additional devices 20a, 20b are mounted, arranged, or fitted on the person 50 for measuring vital signs of said person 50: the first additional device 20a is on the chest 53 and the second additional device 20b is on the wrist, for example on the anterior portion of the wrist (e.g., the portion of the wrist not shown in FIG. 3). It is easier to feel the pulse on the anterior portion of the wrist than on the posterior portion of the wrist. The person 50 has a right upper leg 51 and a right lower leg 52. The body members 51-53 form a kinematic chain.

In FIG. 2, the first predetermined movement to be performed by a user is a quick knee-up, which involves the lower leg 52 and the upper leg 51 of a same leg. For example, the user 50 may select the quick knee-up movement in a user interface of the motion measuring system 5, e.g., a user interface of the computing device 10 (such as a user interface provided by the device 14), thereby causing the motion measuring system 5 to generate or receive a signal which indicates that the first predetermined movement to be performed by the person 50 is a quick knee-up.

A user interface of the motion measuring system 5 may thus instruct the person 50 to perform a movement of one or more body members. The motion measuring system 5 may provide a motion measuring sequence of the chest 53, upper leg 51 and lower leg 52 of the user 50 in the performance of the first predetermined movement by the user 50. In this movement, the chest 53 remains still or almost still during the entire movement, and the upper leg 51 and the lower leg 52 move faster than the chest 53 and are subjected to higher accelerations and decelerations than the chest 53 in each performance of the first predetermined movement.

In FIG. 3, the person 50 has the two additional devices 20a, 20b mounted, arranged, or fitted as in FIG. 2, the difference in FIG. 3 being that the person 50 performs a second predetermined movement which is a slow squat, which involves the lower legs or shanks 52, and the upper legs or thighs 51, and the chest 53. In this movement, the end 57 of the lower legs 52 that connects to the ankle has a known position that remains still or almost still during the entire movement.

In the case of FIG. 2 and FIG. 3, in order to enhance accuracy of the provided motion measuring sequence while minimizing or reducing a negative impact on communications and/or power consumption, the motion measuring system 5 samples fewer data per unit of time by means of the optical sensor 31 of the camera 30 in the second predetermined movement than in the first predetermined movement. In other words, a sampling frequency associated with the optical sensor 31 in the second predetermined movement is lower than in the first predetermined movement.

In some examples, if the optical sensor 31 takes measurements at the measurement frequency, the taken measurements of light are digitized at the digitizing frequency, the digitized measurements of light are processed at the processing frequency and the processed measurements of light are transmitted at the transmission frequency to the computing device 10 which provides the motion measuring sequence, a sampling frequency associated with the optical sensor 31 is dependent on the measurement frequency, the digitizing frequency, the processing frequency, and the transmission frequency. At least one of the measurement frequency, the digitizing frequency, the processing frequency, and the transmission frequency associated with the optical sensor 31 is configured by the camera 30 by modifying a respective predetermined frequency associated with the optical sensor 31.

In some examples, the optical sensor 31 is associated with a predetermined measurement frequency with which the optical sensor 31 measures light, a predetermined digitizing frequency with which the camera 30 digitizes the measurements of light, a predetermined processing frequency with which the camera 30 processes the measurements of light, and a predetermined transmission frequency with which the camera 30 transmits the measurements of light to the computing device 10. The predetermined measurement frequency associated with the optical sensor 31 may be different from the predetermined digitizing frequency associated with the optical sensor 31 and/or from the predetermined processing frequency associated with the optical sensor 31 and/or from the predetermined transmission frequency associated with the optical sensor 31. The predetermined digitizing frequency associated with the optical sensor 31 may be different from the predetermined processing frequency associated with the optical sensor 31 and/or from the predetermined transmission frequency associated with the optical sensor 31. The predetermined processing frequency associated with the optical sensor 31 may be different from the predetermined transmission frequency associated with the optical sensor 31. For the purpose of simplicity of this description, it is considered that the predetermined measurement frequency associated with the optical sensor 31 is equal to the predetermined digitizing frequency associated with the optical sensor 31, equal to the predetermined processing frequency associated with the optical sensor 31, and equal to the predetermined transmission frequency associated with the optical sensor 31.

predetermined measurement frequency=original_sampling_frequency

predetermined digitizing frequency=original_sampling_frequency

predetermined processing frequency=original_sampling_frequency

predetermined transmission frequency=original_sampling_frequency

The original_sampling_frequency is, for example, a default frequency associated with the optical sensor 31. For example, the original_sampling_frequency may be between 30 MHz and 72 MHz (inclusive), for example one of 30 MHz, 35 MHz, 40 MHz, 45 MHz, 50 MHz, 55 MHz, 60 MHz, 65 MHz, 70 MHz, and 72 MHz. The measurement frequency of 30 MHz may be obtained with a camera capturing an image every 200 milliseconds, the camera measuring light with 6 million light sensors in the capture of each image. The measurement frequency of 60 MHz may be obtained with a camera capturing an image every 100 milliseconds, the camera measuring light with 6 million light sensors in the capture of each image. In the following example it is considered that the original_sampling_frequency is 30 MHz.

In some examples, the measurement frequency, the digitizing frequency, the processing frequency, and the transmission frequency associated with the optical sensor 31 are configured by the camera 30 based on one or more of:
the predetermined movement to be performed by the user 50, and
measurements of the motion measurement of the user 50 based on the measurements taken by the optical sensor 31.

The measurement frequency associated with the optical sensor 31 is configured by the camera 30 resulting in an adjustment of the measurement frequency associated with the optical sensor 31. The adjustment of the measurement frequency comprises applying an adjustment value a_{yyy} to the predetermined measurement frequency. The adjustment value a_{yyy} is configured to be applied to the predetermined measurement frequency associated with the optical sensor 31.

In some examples, the adjustment value is a first adjustment value a₁₁₁ of a first plurality of adjustment values a₁₁₁, a₁₁₂, . . . a₁₁ₘ, a₁₂₁, a₁₂₂, . . . a₁ₚₘ. Each adjustment value of the first plurality of adjustment values is associated with one predetermined movement to be performed by the user 50. At least some of the first plurality of adjustment values is configured to be applied to the predetermined measurement frequency associated with the optical sensor 31. In the case of FIG. 2, the first adjustment value a₁₁₁ is associated with the quick knee-up movement.

In some examples, if the predetermined measurement frequency is appropriate for sampling a motion of a movement requiring a high level of motion (e.g., jumping) compared to the quick knee-up movement, then the application of the first adjustment value a₁₁₁ to the predetermined measurement frequency results in a measurement frequency which is lower than the predetermined measurement frequency. In some examples, each adjustment value of the first plurality of adjustment values is a factor configured to multiply the respective predetermined frequency, the first adjustment value a₁₁₁ being, for example, 0.5:

measurement frequency=predetermined measurement frequency·a₁₁₁=30·0.5=15 (MHz)

This results in a measurement frequency associated with the optical sensor 31 of 15 MHz.

In some examples, the adjustment value of the first plurality of adjustment values configured to be applied to the predetermined digitizing frequency associated with the optical sensor 31 and associated with the quick knee-up movement is the first adjustment value a₁₁₁:

digitizing frequency=predetermined digitizing frequency·a₁₁₁

In some examples, the adjustment value of the first plurality of adjustment values configured to be applied to the predetermined digitizing frequency associated with the optical sensor 31 and associated with the quick knee-up movement is lower or higher than the first adjustment value a₁₁₁.

In some examples, the adjustment value of the first plurality of adjustment values configured to be applied to the predetermined processing frequency associated with the optical sensor 31 and associated with the quick knee-up movement is the first adjustment value a₁₁₁:

processing frequency=predetermined processing frequency·a₁₁₁

In some examples, the adjustment value of the first plurality of adjustment values configured to be applied to the predetermined processing frequency associated with the optical sensor 31 and associated with the quick knee-up movement is lower or higher than the first adjustment value a₁₁₁.

In some examples, the adjustment value of the first plurality of adjustment values configured to be applied to the predetermined transmission frequency associated with the optical sensor 31 and associated with the quick knee-up movement is the first adjustment value a₁₁₁:

transmission frequency=predetermined transmission frequency·a₁₁₁

In some examples, the adjustment value of the first plurality of adjustment values configured to be applied to the predetermined transmission frequency associated with the optical sensor 31 and associated with the quick knee-up movement is lower or higher than the first adjustment value a₁₁₁.

The predetermined measurement frequency associated with the one or more additional sensors 21 (or a subset thereof) may have a different value than the predetermined measurement frequency associated with the optical sensor 31. For example, the predetermined measurement frequency associated with the one or more additional sensors 21 may be between 5 Hz and 250 Hz, for example one of 10 Hz, 15 Hz, 25 Hz, 30 Hz, 40 Hz, 45 Hz, 55 Hz, 60 Hz, 70 Hz, 75 Hz, 85 Hz, 90 Hz, 100 Hz, 105 Hz, 110 Hz, 115 Hz, 120 Hz, 125 Hz, 130 Hz, 135 Hz, 140 Hz, 145 Hz, 150 Hz, 155 Hz, 160 Hz, 165 Hz, 170 Hz, 175 Hz, 180 Hz, 185 Hz, 190 Hz, 195 Hz, 200 Hz, 205 Hz, 210 Hz, 215 Hz, 220 Hz, 225 Hz, 230 Hz, 235 Hz, 240 Hz, 245 Hz, or 250 Hz. In the following example it is considered that the predetermined measurement frequency is 50 Hz.

A second adjustment value a₁₁₂ of the first plurality of adjustment values configured to be applied to the predetermined measurement frequency associated with the one or more additional sensors 21 and associated with the quick knee-up movement is, for example, equal to the first adjustment value am:

measurement frequency=predetermined measurement frequency·a₁₁₂=50·0.5=25 (Hz)

In some examples, the adjustment value of the first plurality of adjustment values configured to be applied to the predetermined digitizing frequency associated with the one or more additional sensors 21 and associated with the quick knee-up movement is the second adjustment value a₁₁₂:

digitizing frequency=predetermined digitizing frequency·a₁₁₂

In some examples, the adjustment value of the first plurality of adjustment values configured to be applied to the predetermined digitizing frequency associated with the one or more additional sensors 21 and associated with the quick knee-up movement is lower or higher than the second adjustment value a₁₁₂.

In some examples, the adjustment value of the first plurality of adjustment values configured to be applied to the predetermined processing frequency associated with the one or more additional sensors 21 and associated with the quick knee-up movement is the second adjustment value a₁₁₂:

processing frequency=predetermined processing frequency·a₁₁₂

In some examples, the adjustment value of the first plurality of adjustment values configured to be applied to the predetermined processing frequency associated with the one or more additional sensors 21 and associated with the quick knee-up movement is lower or higher than the second adjustment value a₁₁₂.

In some examples, the adjustment value of the first plurality of adjustment values configured to be applied to the predetermined transmission frequency associated with the one or more additional sensors 21 and associated with the quick knee-up movement is the second adjustment value a₁₁₂:

transmission frequency=predetermined transmission frequency·a₁₁₂

In some examples, the adjustment value of the first plurality of adjustment values configured to be applied to the predetermined transmission frequency associated with the one or more additional sensors 21 and associated with the quick knee-up movement is lower or higher than the second adjustment value a₁₁₂.

In some examples, the measurement frequency associated with the one or more additional sensors 21 and resulting from the adjustment associated with the one or more additional sensors 21 is lower than the measurement frequency associated with the optical sensor 31 and resulting from the adjustment associated with the optical sensor 31, the digitizing frequency associated with the one or more additional sensors 21 and resulting from the adjustment associated with the one or more additional sensors 21 is lower than the digitizing frequency associated with the optical sensor 31 and resulting from the adjustment associated with the optical sensor 31, the processing frequency associated with the one or more additional sensors 21 and resulting from the adjustment associated with the one or more additional sensors 21 is lower than the processing frequency associated with the optical sensor 31 and resulting from the adjustment associated with the optical sensor 31 and the transmission frequency associated with the one or more additional sensors 21 and resulting from the adjustment associated with the one or more additional sensors 21 is lower than the transmission frequency associated with the optical sensor 31 and resulting from the adjustment associated with the optical sensor 31. These examples may be advantageous for achieving an appropriate accuracy of both motion measuring sequence and estimations from measurements of vital signs because providing an accurate motion measuring sequence may require more measurements of light per unit of time compared to a number of measurements of vital signs (e.g., temperature) per unit of time required for obtaining the estimations. Accordingly, in some examples, the one or more adjusted frequencies of the one or more additional sensors (e.g., body member sensors) is lower than the one or more adjusted frequencies associated with the one or more optical sensors.

In a slow squat, such as the slow squat illustrated in FIG. 3, the speed, acceleration and deceleration of body members may be lower than in the quick knee-up. In order to enhance accuracy of the provided motion measuring sequence while minimizing or reducing a negative impact on communications and/or power consumption, the adjustment value of the first plurality of adjustment values associated with the slow squat and with the optical sensor 31 is lower than the first adjustment value am associated with the quick knee-up and with the optical sensor 31. For example, the adjustment value associated with the slow squat and with the optical sensor 31 is 0.25.

In order to enhance accuracy of the provided estimation while minimizing or reducing a negative impact on communications and/or power consumption, the adjustment value of the first plurality of adjustment values associated with the slow squat and with the one or more additional sensors 21 is lower than the second adjustment value a₁₂ associated with the quick knee-up and with the one or more additional sensors 21. For example, the adjustment value associated with the slow squat and with the one or more additional sensors 21 is 0.25.

Although in the previous examples it has been considered that predetermined movements defining higher level of motion of body members (e.g., at least one of: faster, with higher linear acceleration, with higher linear deceleration, with higher variation of linear acceleration, with higher variation of linear deceleration, with higher angular acceleration, with higher angular deceleration, with higher variation of angular acceleration, and with higher variation of angular deceleration) are associated with a higher frequency resulting from the adjustment associated with the particular optical sensor 31 and/or one or more additional sensors 21, in other examples the frequency resulting from the adjustment of the frequency associated with the optical sensor 31 and/or the one or more additional sensors 21 and associated with a predetermined movement having higher level of motion is lower than the frequency resulting from the adjustment of frequency associated with the optical sensor 31 and/or the one or more additional sensors 21 and associated with a predetermined movement having lower level of motion. For example, it may be appropriate to sample movement of a neck at a higher frequency than other more robust body members which move with higher intensity than the neck because the neck is relatively weaker and slightly wrong movements of the neck may have a relatively high impact on, for example, rehabilitation movements.

In some examples, the adjustment value of the first plurality of adjustment values is determined based on data indicative of the first plurality of adjustment values, the predetermined movement to be performed by the user 50, and the sensor associated with the adjustment value.

The data indicative of the first plurality of adjustment values may be stored in a memory, for example, in the at least one memory 12 of the one or more additional devices 20a-20n and/or in the at least one memory 33 of the camera 30 and/or in the at least one memory 23 of an additional sensor 21. In some examples, the camera 30 and/or the one or more additional devices 20a-20n of the motion measuring system 5 receive the data indicative of the first plurality of adjustment values from a computing device, such as the computing device 10. The data indicative of the first plurality of adjustment values may comprise the first plurality of adjustment values and relationships of each adjustment value of the first plurality of adjustment values with one predetermined movement and with one sensor of the optical sensor 31 and the one or more additional sensors 21.

In some examples, the camera 30 determines an adjustment value based on the first plurality of adjustment values stored in the memory 33 of the camera and based on a predetermined movement to be performed by the user 50. For example, the camera 30 may determine the first adjustment value based on the quick knee-up movement.

The motion measuring may be performed by methods already known in the art based on the measurements of light taken by the camera 30, for example, from images captured by the optical sensor 31, data indicative of lengths of body members of the user 50, and data indicative of position and orientation of reference points of the body of the user 50.

In some examples, a computing device, such as the computing device 10, obtains data indicative of an adjustment value of the first plurality of adjustment values, the adjustment value being associated with a predetermined movement to be performed by the user 50 and a sensor of the optical sensor 31 and the one or more additional sensors 21. The camera 30 and/or the one or more additional devices 20a-20n receive the data indicative of the adjustment value. For example, the camera 30 may determine the first adjustment value based on received data indicative of the first adjustment value. In some examples, the data indicative of the first adjustment value is the first adjustment value.

The determination of an adjustment value of the first plurality of adjustment values may comprise retrieving the adjustment value associated with the predetermined movement to be performed by the user 50 and the sensor associated with the adjustment value (e.g., the sensor which frequency is to be adjusted with the determined adjustment value). The retrieving is performed, for example, by means of executing an instruction for retrieving data stored in a memory, the instruction comprising data indicative of the predetermined movement to be performed by the user 50 and the sensor associated with the adjustment value. The instruction is, for example, a query for retrieving data from a database stored in a memory.

In some examples, each adjustment value of the first plurality of adjustment values is further associated with the user 50 and a parameter indicative of a level of motion of the one or more body members as measured based on measurements of light of the optical sensor 31 in a previous performance of the predetermined movement. For example, prior to determining and/or updating the first adjustment value, the user 50 may perform one or more repetitions of a quick knee-up. The optical sensor 31 obtains light measurements of the movement of the body members in the repetitions. One or more levels of motion of the body members are obtained based on the light measurements. If the user 50 has, for example, an injury in the back, the movement of the body members in the one or more repetitions may be slower compared to the same movement performed by the same user 50 without the back injury. Accordingly, accuracy of the provided motion measuring sequence may barely decrease by decreasing the measurement frequency, the digitizing frequency, the processing frequency and/or the transmission frequency associated with the optical sensor 31 compared with an accuracy obtained with the respective measurement frequency, digitizing frequency, processing frequency and/or transmission frequency associated with the optical sensor 31.

In some examples, the measurement frequency, the digitizing frequency, the processing frequency and/or the transmission frequency associated with the optical sensor 31 and/or the one or more additional sensors 21 is configured based on measurements of light of the optical sensor 31. In particular, the at least one frequency associated with the optical sensor 31 and/or the one or more additional sensors 21 is configured by applying an adjustment value of the second plurality of adjustment values to the respective predetermined frequency associated with the optical sensor 31 and/or the one or more additional sensors 21. Each adjustment value of the second plurality of adjustment values is associated with a level of motion of the one or more body members as measured based on measurements of light of the optical sensor 31, the level of motion being within a predetermined motion range. An example is explained below.

In some examples, the user 50 performs the predetermined movement slower or faster than it is expected according to the first plurality of adjustment values associated with the predetermined movement. For example, the user 50 performs the quick knee-up movement raising the leg slowly (relative to an expectation or predefined threshold). Since the first plurality of adjustment values do not take into account this situation, an adjustment of the measurement frequency, digitizing frequency, processing frequency and/or transmission frequency associated with the optical sensor 31 by applying the corresponding adjustment value of the first plurality of adjustment values will result in a frequency higher than required or desired. To overcome this potential limitation of the first plurality of adjustment values, an adjustment value of the second plurality of adjustment values may be applied to the respective predetermined frequency. In this case, both the first adjustment value a₁₁₁ of the first plurality of adjustment values and a third adjustment value a₂₂₁ of the second plurality of adjustment values are applied to the respective predetermined frequency, e.g. the predetermined measurement frequency:

measurement frequency=predetermined measurement freq·a₁₁₁·a₂₂₁=30·0.5·0.5=7.5 MHz

Accordingly, in some examples, as the person 50 carries out relevant movement/s or exercise/s (e.g., based on instructions transmitted via the user interface), additional adjustment of one or more frequencies of one or more of the sensors may be performed. For example, additional adjustments may be made by the motion measuring system 5 to further reduce the power consumption of at least one of the sensors.

The additional adjustment may be based on a measured level of motion. A magnitude of the additional adjustment may be based on the measured level of motion and/or a range of motion associated with the movement.

The motion measuring system 5 may thus dynamically and selectively adjust frequencies. Frequencies may be repeatedly adjusted during operation of the motion measuring system 5, e.g., to maintain at least a threshold motion measuring accuracy level while increasing a life of a battery of one or more of the sensors (e.g., the camera 30 or one or more of the additional sensors 21).

If the level of motion of the one or more body members decreases a lot, for example, because the user 50 remains still, then the level of motion of the one or more body members is within a predetermined motion range associated with the one or more body members being motionless. In this case, waste of power consumed by the optical sensor 31 may be minimized by applying a fourth adjustment value a₂₁₂ of the second plurality of adjustment values to the predetermined transmission frequency associated with the optical sensor 31, the fourth adjustment value a₂₁₂ corresponding to no transmission. For example, the fourth adjustment value a₂₁₂ may be zero:

transmission frequency=predetermined transmission frequency·a₂₁₂=30·0=0 MHz

Upon setting the transmission frequency to zero, the optical sensor 31 may transmit, to the computing device 10, data indicative of at least temporal no transmission of measurements.

Although specific examples are described herein, it will be evident that various modifications and changes may be made to these examples without departing from the broader spirit and scope of the disclosure. Accordingly, the specification and drawings are to be regarded in an illustrative rather than a restrictive sense. The accompanying drawings that form a part hereof show by way of illustration, and not of limitation, specific examples in which the subject matter may be practiced. The examples illustrated are described in sufficient detail to enable those skilled in the art to practice the teachings disclosed herein. Other examples may be utilized and derived therefrom, such that structural and logical substitutions and changes may be made without departing from the scope of this disclosure. This detailed description, therefore, is not to be taken in a limiting sense, and the scope of various examples is defined only by the appended claims, along with the full range of equivalents to which such claims are entitled.

Such examples of the inventive subject matter may be referred to herein, individually or collectively, by the term “example” merely for convenience and without intending to voluntarily limit the scope of this application to any single example or concept if more than one is in fact disclosed. Thus, although specific examples have been illustrated and described herein, it should be appreciated that any arrangement calculated to achieve the same purpose may be substituted for the specific examples shown. This disclosure is intended to cover any and all adaptations or variations of various examples. Combinations of the above examples, and other examples not specifically described herein, will be apparent to those of skill in the art upon reviewing the above description.

Some portions of the subject matter discussed herein may be presented in terms of algorithms or symbolic representations of operations on data stored as bits or binary digital signals within a machine memory (e.g., a computer memory). Such algorithms or symbolic representations are examples of techniques used by those of ordinary skill in the data processing arts to convey the substance of their work to others skilled in the art. As used herein, an “algorithm” is a self-consistent sequence of operations or similar processing leading to a desired result. In this context, algorithms and operations involve physical manipulation of physical quantities. Typically, but not necessarily, such quantities may take the form of electrical, magnetic, or optical signals capable of being stored, accessed, transferred, combined, compared, or otherwise manipulated by a machine. It is convenient at times, principally for reasons of common usage, to refer to such signals using words such as “data,” “content,” “bits,” “values,” “elements,” “symbols,” “characters,” “terms,” “numbers,” “numerals,” or the like. These words, however, are merely convenient labels and are to be associated with appropriate physical quantities.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or any suitable combination thereof), registers, or other machine components that receive, store, transmit, or display information. Furthermore, unless specifically stated otherwise, the terms “a” and “an” are herein used, as is common in patent documents, to include one or more than one instance. As used herein, the conjunction “or” refers to a non-exclusive “or,” unless specifically stated otherwise.

Unless the context clearly requires otherwise, throughout the description and the claims, the words “comprise,” “comprising,” and the like are to be construed in an inclusive sense, as opposed to an exclusive or exhaustive sense, e.g., in the sense of “including, but not limited to.” As used herein, the terms “connected,” “coupled,” or any variant thereof means any connection or coupling, either direct or indirect, between two or more elements; the coupling or connection between the elements can be physical, logical, or a combination thereof. Additionally, the words “herein,” “above,” “below,” and words of similar import, when used in this application, refer to this application as a whole and not to any particular portions of this application. Where the context permits, words using the singular or plural number may also include the plural or singular number, respectively. The word “or” in reference to a list of two or more items, covers all of the following interpretations of the word: any one of the items in the list, all of the items in the list, and any combination of the items in the list.

Although some examples may include a particular sequence of operations, the sequence may be altered without departing from the scope of the present disclosure. For example, some of the operations depicted may be performed in parallel or in a different sequence that does not materially affect the functions as described in the examples. In other examples, different components of an example device or system that implements an example method may perform functions at substantially the same time or in a specific sequence.

As used herein, the term “processor” may refer to any one or more circuits or virtual circuits (e.g., a physical circuit emulated by logic executing on an actual processor) that manipulates data values according to control signals (e.g., commands, opcodes, machine code, control words, macroinstructions, etc.) and which produces corresponding output signals that are applied to operate a machine. A processor may, for example, include at least one of a Central Processing Unit (CPU), a Reduced Instruction Set Computing (RISC) Processor, a Complex Instruction Set Computing (CISC) Processor, a Graphics Processing Unit (GPU), a Digital Signal Processor (DSP), a Tensor Processing Unit (TPU), a Neural Processing Unit (NPU), a Vision Processing Unit (VPU), a Machine Learning Accelerator, an Artificial Intelligence Accelerator, an Application Specific Integrated Circuit (ASIC), a Field Programmable Gate Array (FPGA), a Radio-Frequency Integrated Circuit (RFIC), a Neuromorphic Processor, a Quantum Processor, or any combination thereof. A processor may be a multi-core processor having two or more independent processors (sometimes referred to as “cores”) that may execute instructions contemporaneously. Multi-core processors may contain multiple computational cores on a single integrated circuit die, each of which can independently execute program instructions in parallel. Parallel processing on multi-core processors may be implemented via architectures like superscalar, VLIW, vector processing, or SIMD that allow each core to run separate instruction streams concurrently. A processor may be emulated in software, running on a physical processor, as a virtual processor or virtual circuit. The virtual processor may behave like an independent processor but is implemented in software rather than hardware.

The various operations of example methods described herein may be performed, at least partially, by one or more processors that are temporarily configured (e.g., by software) or permanently configured to perform the relevant operations. Whether temporarily or permanently configured, such processors may constitute processor-implemented modules/components that operate to perform one or more operations or functions. The modules/components referred to herein may, in some examples, comprise processor-implemented modules/components.

Similarly, the methods described herein may be at least partially processor-implemented. For example, at least some of the operations of a method may be performed by one or more processors or processor-implemented modules/components. The performance of certain of the operations may be distributed among the one or more processors, not only residing within a single machine, but deployed across a number of machines. In some examples, the processor or processors may be located in a single location (e.g., within a home environment, an office environment, or a server farm), while in other examples the processors may be distributed across a number of locations.

Examples may be implemented in digital electronic circuitry, or in computer hardware, firmware, or software, or in combinations of them. Examples may be implemented using a computer program product, e.g., a computer program tangibly embodied in an information carrier, e.g., in a machine-readable medium for execution by, or to control the operation of, data processing apparatus, e.g., a programmable processor, a computer, or multiple computers.

A computer program can be written in any form of programming language, including compiled or interpreted languages, and it can be deployed in any form, including as a standalone program or as a module, subroutine, or other unit suitable for use in a computing environment. A computer program can be deployed to be executed on one computer or on multiple computers at one site or distributed across multiple sites and interconnected by a communication network.

When an ordinal number (such as “first”, “second”, “third” and so on) is used as an adjective before a term or expression, that ordinal number is used (unless expressly specified otherwise) merely to indicate a particular feature, such as to distinguish that particular feature from another feature that is described by the same term or expression or by a similar term or expression. For example, a “first widget” may be so named merely to distinguish it from, e.g., a “second widget”. Thus, the mere usage of the ordinal numbers “first” and “second” before the term “widget” does not indicate any other relationship between the two widgets, and likewise does not indicate any other characteristics of either or both widgets. For example, the mere usage of the ordinal numbers “first” and “second” before the term “widget” does not indicate that either widget comes sequentially before or after any other in order or location; does not indicate that either widget occurs or acts before or after any other in time; and does not indicate that either widget ranks above or below any other, as in importance or quality. In addition, the mere usage of ordinal numbers does not define a numerical limit to the features identified with the ordinal numbers. For example, the mere usage of the ordinal numbers “first” and “second” before the term “widget” does not indicate that there must be no more than two widgets and the mere usage of the ordinal number “second” before the term “widget” does not indicate that there must be a “first widget”.

In view of the above-described implementations of subject matter this application discloses the following list of examples, wherein one feature of an example in isolation or more than one feature of an example, taken in combination and, optionally, in combination with one or more features of one or more further examples are further examples also falling within the disclosure of this application.

Example 1 is a method for reducing a power consumption of a motion measuring system, the motion measuring system comprising a plurality of sensors and at least one computing device in communication with the plurality of sensors, the plurality of sensors comprising one or more optical sensors and one or more body member sensors, the one or more optical sensors arranged at a distance from a subject so that the one or more optical sensors receive light from one or more body members of the subject, and the one or more body member sensors arranged on at least one body member of the subject, the method comprising: using a user interface provided by the at least one computing device to instruct the subject to perform a movement of the one or more body members; performing an adjustment of one or more frequencies of one or more of the plurality of sensors to reduce a power consumption of at least one of the plurality of sensors to thereby reduce the power consumption of the motion measuring system, wherein the adjustment is performed based at least in part on the movement which the subject is instructed to perform; and during or subsequent to performing the adjustment of the one or more frequencies, measuring motion of the subject using the one or more optical sensors of the motion measuring system.

In Example 2, the subject matter of Example 1 includes, wherein the adjustment of the one or more frequencies comprises adjusting one or more frequencies of the one or more optical sensors.

In Example 3, the subject matter of any of Examples 1-2 includes, wherein the adjustment of the one or more frequencies comprises adjusting one or more frequencies of the one or more body member sensors.

In Example 4, the subject matter of any of Examples 1-3 includes, wherein the adjustment of the one or more frequencies comprises adjusting one or more frequencies of the one or more optical sensors and one or more frequencies of the one or more body member sensors.

In Example 5, the subject matter of Example 4 includes, wherein the one or more adjusted frequencies of the one or more body member sensors is lower than the one or more adjusted frequencies of the one or more optical sensors.

In Example 6, the subject matter of any of Examples 1-5 includes, wherein the adjustment of the one or more frequencies comprises adjusting the one or more frequencies based at least in part on a level of motion associated with the subject, wherein the association is determined by a previously recorded measurement of the subject by the motion measuring system.

In Example 7, the subject matter of any of Examples 1-6 includes, wherein the measuring of the motion of the subject is performed during the adjustment of the one or more frequencies.

In Example 8, the subject matter of Example 7 includes, subsequent to the adjustment of the one or more frequencies, performing an additional adjustment of the one or more frequencies of one or more of the plurality of sensors during the performance of the movement by the subject to further reduce the power consumption of the at least one of the plurality of sensors.

In Example 9, the subject matter of Example 8 includes, wherein the additional adjustment is based at least in part on a measured level of motion associated with the subject.

In Example 10, the subject matter of any of Examples 1-9 includes, adjusting a transmission frequency of the one or more optical sensors such that substantially no transmission of information from the one or more optical sensors to the at least one computing device occurs when the one or more body members are motionless.

In Example 11, the subject matter of Example 10 includes, detecting, by the motion measuring system, that the one or more body members are motionless; in response to detecting that the one or more body members is motionless, halting the measuring of the motion; and providing an alert of a fault condition of the motion measuring system.

In Example 12, the subject matter of any of Examples 1-11 includes, wherein the motion measuring system comprises a camera that includes at least one of the one or more optical sensors.

In Example 13, the subject matter of any of Examples 1-12 includes, wherein at least one of the one or more body member sensors comprises a vital sign sensor.

In Example 14, the subject matter of Example 13 includes, wherein the vital sign sensor comprises a respiration rate sensor, a body temperature sensor, a pulse rate sensor, or a combination of two or more thereof.

In Example 15, the subject matter of Example 14 includes, wherein the one or more frequencies are repeatedly adjusted during operation of the motion measuring system.

In Example 16, the subject matter of any of Examples 14-15 includes, wherein the one or more frequencies are repeatedly adjusted to maintain at least a threshold motion measuring accuracy level while increasing a life of a battery of one or more of the plurality of sensors.

In Example 17, the subject matter of any of Examples 1-16 includes, wherein the one or more optical sensors is powered by at least one battery.

In Example 18, the subject matter of any of Examples 1-17 includes, wherein the one or more body member sensors is powered by at least one battery.

Example 19 is a motion measuring system comprising a plurality of sensors and at least one computing device in communication with the plurality of sensors, the plurality of sensors comprising one or more optical sensors and one or more body member sensors, and the motion measuring system being configured to perform operations comprising: using a user interface provided by the at least one computing device to instruct a subject to perform a movement of one or more body members of the subject; performing an adjustment of one or more frequencies of one or more of the plurality of sensors to reduce a power consumption of at least one of the plurality of sensors to thereby reduce a power consumption of the motion measuring system, wherein the adjustment is performed based at least in part on the movement which the subject is instructed to perform; and during or subsequent to performing the adjustment of the one or more frequencies, measuring motion of the subject using the one or more optical sensors, wherein, during the measuring of the motion of the subject, the one or more optical sensors is arranged at a distance from the subject so that the one or more optical sensors receive light from the one or more body members of the subject, and the one or more body member sensors is arranged on at least one body member of the subject.

Example 20 is a non-transitory computer readable medium comprising instructions that, when executed by at least one computer processor, cause the at least one computer processor to perform operations comprising: in a motion measuring system comprising a plurality of sensors and at least one computing device in communication with the plurality of sensors, the plurality of sensors comprising one or more optical sensors and one or more body member sensors, the one or more optical sensors arranged at a distance from a subject so that the one or more optical sensors receive light from one or more body members of the subject, and the one or more body member sensors arranged on at least one body member of the subject, using a user interface provided by the at least one computing device to instruct the subject to perform a movement of the one or more body members; performing an adjustment of one or more frequencies of one or more of the plurality of sensors to reduce a power consumption of at least one of the plurality of sensors to thereby reduce a power consumption of the motion measuring system, wherein the adjustment is performed based at least in part on the movement which the subject is instructed to perform; and during or subsequent to performing the adjustment of the one or more frequencies, measuring motion of the subject using the one or more optical sensors of the motion measuring system.

Example 21 is at least one machine-readable medium including instructions that, when executed by processing circuitry, cause the processing circuitry to perform operations to implement any of Examples 1-20.

Example 22 is an apparatus comprising means to implement any of Examples 1-20.

Example 23 is a system to implement any of Examples 1-20.

Example 24 is a method to implement any of Examples 1-20.

1. A method for reducing a power consumption of a motion measuring system, the motion measuring system comprising a plurality of sensors and at least one computing device in communication with the plurality of sensors, the plurality of sensors comprising one or more optical sensors and one or more body member sensors, the one or more optical sensors arranged at a distance from a subject so that the one or more optical sensors receive light from one or more body members of the subject, and the one or more body member sensors arranged on at least one body member of the subject, the method comprising:
using a user interface provided by the at least one computing device to instruct the subject to perform a movement of the one or more body members;
performing an adjustment of one or more frequencies of one or more of the plurality of sensors to reduce a power consumption of at least one of the plurality of sensors to thereby reduce the power consumption of the motion measuring system, wherein the adjustment is performed based at least in part on the movement which the subject is instructed to perform; and
during or subsequent to performing the adjustment of the one or more frequencies, measuring motion of the subject using the one or more optical sensors of the motion measuring system.

2. The method of claim 1, wherein the adjustment of the one or more frequencies comprises adjusting one or more frequencies of the one or more optical sensors.

3. The method of claim 1, wherein the adjustment of the one or more frequencies comprises adjusting one or more frequencies of the one or more body member sensors.

4. The method of claim 1, wherein the adjustment of the one or more frequencies comprises adjusting one or more frequencies of the one or more optical sensors and one or more frequencies of the one or more body member sensors.

5. The method of claim 4, wherein the one or more adjusted frequencies of the one or more body member sensors is lower than the one or more adjusted frequencies of the one or more optical sensors.

6. The method of claim 1, wherein the adjustment of the one or more frequencies comprises adjusting the one or more frequencies based at least in part on a level of motion associated with the subject, wherein the association is determined by a previously recorded measurement of the subject by the motion measuring system.

7. The method of claim 1, wherein the measuring of the motion of the subject is performed during the adjustment of the one or more frequencies.

8. The method of claim 7, further comprising, subsequent to the adjustment of the one or more frequencies, performing an additional adjustment of the one or more frequencies of one or more of the plurality of sensors during the performance of the movement by the subject to further reduce the power consumption of the at least one of the plurality of sensors.

9. The method of claim 8, wherein the additional adjustment is based at least in part on a measured level of motion associated with the subject.

10. The method of claim 1, further comprising adjusting a transmission frequency of the one or more optical sensors such that substantially no transmission of information from the one or more optical sensors to the at least one computing device occurs when the one or more body members are motionless.

11. The method of claim 10, further comprising:
detecting, by the motion measuring system, that the one or more body members are motionless;
in response to detecting that the one or more body members is motionless, halting the measuring of the motion; and
providing an alert of a fault condition of the motion measuring system.

12. The method of claim 1, wherein the motion measuring system comprises a camera that includes at least one of the one or more optical sensors.

13. The method of claim 1, wherein at least one of the one or more body member sensors comprises a vital sign sensor.

14. The method of claim 13, wherein the vital sign sensor comprises a respiration rate sensor, a body temperature sensor, a pulse rate sensor, or a combination of two or more thereof.

15. The method of claim 14, wherein the one or more frequencies are repeatedly adjusted during operation of the motion measuring system.

16. The method of claim 14, wherein the one or more frequencies are repeatedly adjusted to maintain at least a threshold motion measuring accuracy level while increasing a life of a battery of one or more of the plurality of sensors.

17. The method of claim 1, wherein the one or more optical sensors is powered by at least one battery.

18. The method of claim 1, wherein the one or more body member sensors is powered by at least one battery.

19. A motion measuring system comprising a plurality of sensors and at least one computing device in communication with the plurality of sensors, the plurality of sensors comprising one or more optical sensors and one or more body member sensors, and the motion measuring system being configured to perform operations comprising:
using a user interface provided by the at least one computing device to instruct a subject to perform a movement of one or more body members of the subject;
performing an adjustment of one or more frequencies of one or more of the plurality of sensors to reduce a power consumption of at least one of the plurality of sensors to thereby reduce a power consumption of the motion measuring system, wherein the adjustment is performed based at least in part on the movement which the subject is instructed to perform; and
during or subsequent to performing the adjustment of the one or more frequencies, measuring motion of the subject using the one or more optical sensors, wherein, during the measuring of the motion of the subject, the one or more optical sensors is arranged at a distance from the subject so that the one or more optical sensors receive light from the one or more body members of the subject, and the one or more body member sensors is arranged on at least one body member of the subject.

20. A non-transitory computer readable medium comprising instructions that, when executed by at least one computer processor, cause the at least one computer processor to perform operations comprising:
in a motion measuring system comprising a plurality of sensors and at least one computing device in communication with the plurality of sensors, the plurality of sensors comprising one or more optical sensors and one or more body member sensors, the one or more optical sensors arranged at a distance from a subject so that the one or more optical sensors receive light from one or more body members of the subject, and the one or more body member sensors arranged on at least one body member of the subject, using a user interface provided by the at least one computing device to instruct the subject to perform a movement of the one or more body members;
performing an adjustment of one or more frequencies of one or more of the plurality of sensors to reduce a power consumption of at least one of the plurality of sensors to thereby reduce a power consumption of the motion measuring system, wherein the adjustment is performed based at least in part on the movement which the subject is instructed to perform; and
during or subsequent to performing the adjustment of the one or more frequencies, measuring motion of the subject using the one or more optical sensors of the motion measuring system.
